SEQUENCE LISTING (19 sequences)

SEQ ID NO: 1 - protein, 5 aa, synthetic construct
GYYMN

SEQ ID NO: 2 - protein, 16 aa, synthetic construct
VIGINGATYYASWAKG

SEQ ID NO: 3 - skipped

SEQ ID NO: 4 - protein, 111 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 5 - protein, 440 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 441 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 13 aa, synthetic construct
QASQSVYHNTYLA

SEQ ID NO: 8 - protein, 7 aa, synthetic construct
DASTLAS

SEQ ID NO: 9 - protein, 13 aa, synthetic construct
LGSYDCTNGDCFV

SEQ ID NO: 10 - protein, 113 aa, synthetic construct
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 11 - protein, 219 aa, synthetic construct
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 17 aa, synthetic construct
KTTVYLQMNSLRAEDTA

SEQ ID NO: 13 - protein, 10 aa, synthetic construct
PEVKFNWYVD

SEQ ID NO: 14 - protein, 16 aa, synthetic construct
ALHNHYTQKSLSLSPG

SEQ ID NO: 15 - protein, 17 aa, synthetic construct
DRVTINCQASQSVYHNT

SEQ ID NO: 16 - protein, 18 aa, synthetic construct
VPKQLIYDASTLASGVPS

SEQ ID NO: 17 - protein, 19 aa, synthetic construct
DNALQSGNSQESVTEQDSK

SEQ ID NO: 18 - protein, 17 aa, synthetic construct
KHKVYACEVTHQGLSSP

SEQ ID NO: 19 - protein, 18 aa, synthetic construct
DSKDSTYSLSSTLTLSKA

(19) United States
(12) Patent Application Publication
(10) Pub. No.: US 2026/0226098 A1
(43) Pub. Date: Aug. 6, 2026

(54) PURIFICATION

(71) Applicant: H. LUNDBECK A/S, VALBY (DK)

(72) Inventors: Sam MARZOLF, Bothell, WA (US); Ethan Wayne OJALA, Bothell, WA (US); Mantas MALISAUSKAS, Copenhagen (DK)

(21) Appl. No.: 19/387,758

(22) Filed: Nov. 13, 2025

Related U.S. Application Data
(60) Provisional application No. 63/720,471, filed on Nov. 14, 2024.
(60) Provisional application No. 63/855,595, filed on Aug. 1, 2025.

(51) Int. Cl. C07K 1/18; C07K 1/22; C07K 16/18

(52) U.S. Cl. CPC C07K 1/18; C07K 1/22; C07K 16/18; C07K 2317/24

(57) ABSTRACT

The present invention relates to methods for isolating a product and/or reducing impurities such as Host Cell Proteins (HCP), aggregates and residual Protein A (rPA) from a load fluid comprising a FC-containing proteins.

### RELATED APPLICATIONS

The present invention relates to and claims benefit of priority to U.S. Provisional Application No. 63/720,471, filed on Nov. 14, 2024, and U.S. Provisional Application No. 63/855,595, filed on Aug. 1, 2025, the contents of both are which are incorporated by reference in their entirety herein.

### RELATED APPLICATIONS

The contents of the electronic sequence listing (1297-WO-PCT-SQ-listing.xml; Size: 18,126 bytes; and Date of Creation: Sep. 5, 2025) is herein incorporated by reference in its entirety.

### FIELD OF THE INVENTION

The invention relates to methods for isolating a product and/or reducing impurities such as aggregates, host cell proteins (HCP), and residual protein A (rPA) from a load fluid comprising the product, preferably an Fc-containing monoclonal antibody such as Eptinezumab, by passing the load fluid through one or more chromatographic columns.

### BACKGROUND OF THE INVENTION

The present invention relates to protein purification and in particular methods for purifying a protein bound to one or more chromatographic resins and subsequently collecting the purified protein in the eluate.

By use of recombinant technology, many proteins, such as therapeutic antibodies, are cultured in eukaryotic or prokaryotic host cell lines engineered to express the protein. The use of the desired recombinant protein for pharmaceutical applications is generally contingent on being able to reliably recover adequate levels of the protein from impurities such as host cell proteins, protein variants, and compounds from the culture medium.

Conventional protein purification methods are designed to separate the protein of interest from impurities based on differences in size, charge, solubility, and degree of hydrophobicity. Such methods include chromatographic methods such as affinity chromatography, ion exchange chromatography, size exclusion chromatography, hydrophobic interaction chromatography, immobilized metal affinity chromatography, and hydroxyapatite chromatography. These methods often employ a chromatographic resin that can be designed to selectively adhere either the protein of interest or the impurities. In the bind-elute mode, the desired protein selectively binds to the chromatographic resin and is differentially eluted from the medium by different solvents. In the flow-through mode, the impurities specifically bind to the chromatographic resin while the protein of interest does not, thus allowing the recovery of the desired protein in the “flow-through.”

Current methods for the purification of proteins, such as antibodies, include two or more chromatographic steps. For example, the first step in the protein purification protocol can involve an affinity chromatography step that utilizes a specific interaction between the protein of interest and an immobilized resin. Not only the specific chromatographic columns used have an impact on reducing the impurities, but also the wash and elution conditions. The inventors of the present invention have found that using an Anion Exchange Chromatographic method results in surprisingly high purity and low amounts of host cell proteins, in particular when applied to the antibodies named herein. In Certain embodiments the Anion Exchange Chromatography is preceded by purification using Cation Exchange Chromatography and Protein A Chromatography.

### SUMMARY OF THE INVENTION

The present invention relates to a method for reducing impurities in an eluate comprising a product, the method comprising:
(a) providing a load fluid comprising a product and one or more impurities, wherein the product is an Fc-containing monoclonal antibody,
(b) applying the load fluid in a) to a Protein A chromatography column under conditions suitable for binding the product,
(c) applying the load fluid from b) to a Cation Exchange chromatography column under conditions suitable for binding the product, and
(d) applying the load fluid from c) to an Anion Exchange chromatography column under conditions suitable for binding impurities, to reduce the percentage of aggregates to 0.2-0.5% or 0.1-0.5%.

The invention further relates to the purification of antibodies and a composition comprising said antibodies, and in particular monoclonal antibodies binding to Calcitonin Gene Related Peptide (CGRP), and in particular Eptinezumab as defined further herein below.

### DESCRIPTION OF THE INVENTION

The present invention provides methods for purifying and recovering a product from a load fluid containing one or more impurities using a purification method that includes an anion chromatographic step. The invention can be applied to the large-scale preparation of proteins for therapeutic and/or diagnostic purposes.

In order for the present invention to be more readily understood, certain terms as used herein are defined. Additional definitions are set forth throughout the detailed description.

The term “product” refers to a molecule produced by a natural process (e.g. through expression in a mammalian cell such a CHO cell). The term “product” includes a protein, e.g., a therapeutic protein, and in particularly monoclonal antibodies able to bind a protein A resin through its Fc-domain, such as the antibody named Eptinezumab as further defined herein below. The terms “product” and “protein of interest” are used interchangeably.

The term “conditioned culture medium” as used herein refers to the supernatant that is generated from the removal of cells and cellular debris by a separation method, such as centrifugation and/or microfiltration, from cell culture medium that has been exposed to host cells, which secrete desired products. Conditioned selected nutrients (e.g. vitamins, amino acids, cofactors, and minerals); additional growth factors/supplements including insulin; and additional exogenous, or host cell proteins and impurities. The term conditioned culture medium includes clarified conditioned medium, filtered conditioned medium, and conditioned cell culture medium.

The term “load fluid” refers to a liquid containing the product (in particular Eptinezumab) to be isolated and one or more impurities. A load fluid contacts a chromatographic resin (e.g., is passed through a chromatographic column) under the operating conditions of the invention described below. In a first step of the invention, the Protein A Chromatographic column may be loaded (load fluid) with conditioned culture medium described herein above in which CHO cells have been expressing and produced Eptinezumab in a bioreactor or bioprocess.

The term “impurity” refers to any foreign or undesirable molecule that is present in a solution such as a load fluid. An impurity can be a biological macromolecule such as DNA, RNA, or a protein. Impurities include, for example, undesirable protein variants and fragments which may be from the protein of interest (Eptinezumab), such as aggregated proteins, misfolded proteins, high molecular weight species, low molecular weight species and fragments, and deamidated species from the protein of interest such as Eptinezumab; other proteins from host cells that secrete the protein being purified, host cell DNA, components from the cell culture medium, molecules that are part of an absorbent used for affinity chromatography that leach into a sample during prior purification steps, for example, Protein A; an endotoxin; a nucleic acid; a virus, or a fragment of any of the forgoing.

The term “resin” refers to an affinity matrix or resin that can undergo a ligand-biomacromolecule interaction with a product to be isolated during a macromolecular separation process. The resin is preferably a Capto S impact for the Cation Exchange chromatography having a resin composed of agarose base with a polymer ligand of pyrrolidone and sulfonate, and for the Anion Exchange Chromatography the resin is composed of a base material of hydroxylated methacrylic polymer beads that have been functionalized with proprietary primary amine (NH2) strong anion exchange groups such as Toyopearl NH2-750F.

The term “host cell proteins” (HCP) refers to non-product proteins produced by a host cell during cell culture or fermentation. Accordingly, in some embodiments, an eluate containing a product has HCPs present in less than 100 parts per million (ppm) HCPs (e.g., less than about 50 ppm, or less than about 20 ppm). HCP composition is extremely heterogeneous and dependent on the protein product and purification procedure used. Prior to any marketing approval of a biological product for therapeutic use, the level of contaminating proteins (such as HCPs) in the product must be quantitatively measured according to the ICH and FDA guidelines. Host cell proteins can be measured using commercial kits such as ELISA HCP kits from Cygnus e.g. CHO HCP ELISA.

The term “residual protein A” (rPA) is an impurity that comes from a Protein A chromatographic step. rPA is efficiently reduced to below 2 ppm using the Cation Exchange and/or the Anion Exchange Chromatography of the invention. Residual protein A can be measured by a commercial kit such as Cygnus Protein A ELISA Kits (F050H, F400, F400Z) and Protein A Mix-N-Go™ ELISA Kits (F600, F610, F740, F910, F950, F965).

The term “protein” as used herein refers to one or more polypeptides that can function as a unit. The term “polypeptide” as used herein refers to a sequential chain of amino acids linked together via peptide bonds. A therapeutic protein can be, for example, a secreted protein. Therapeutic proteins include antibodies, antigen-binding fragments of antibodies, some of which are described in more detail herein below.

The term “antibody” refers to any immunoglobulin and encompasses any polypeptide comprising an antigen-binding site. The term includes, but is not limited to, polyclonal, monoclonal, monospecific, polyspecific, non-specific, humanized, human, single-chain, chimeric, synthetic, recombinant, hybrid, mutated, grafted, and in vitro generated antibodies.

In further embodiments of the invention, the product is an antibody that has a CH 2/CH 3 region and therefore is amenable to purification by Protein A chromatography. The term “CH2/CH3 region” refers to those amino acid residues in the Fc region of an immunoglobulin molecule that interact with Protein A. In particular, the antibody of the invention is a monoclonal Fc-containing antibody capable of binding to a protein A ligand.

Examples of Fc-containing monoclonal antibodies of the invention includes the below described antibodies binding to Calcitonin Gene Related Peptide (CGRP) and in particular Eptinezumab as further described herein below.

Calcitonin Gene Related Peptide (CGRP) is produced as a multifunctional neuropeptide of 37 amino acids in length. Two forms of CGRP, the CGRP-alpha and CGRP-beta forms, exist in humans and have similar activities. CGRP-alpha and CGRP-beta differ by three amino acids in humans, and are derived from different genes. The CGRP family of peptides includes amylin, adrenomedullin, and calcitonin, although each has distinct receptors and biological activities. Doods, H., Curr. Op. Invest. Drugs, 2(9):1261-68 (2001).

Migraines are neurovascular disorder affecting approximately 10% of the adult population in the U.S., and are typically accompanied by intense headaches. Approximately 20-30% of migraine sufferers experience aura, comprising focal neurological phenomena that precede and/or accompany the event. CGRP is believed to play a prominent role in the development of migraines. For example, plasma concentrations of CGRP were identified elevated in jugular venous blood during the headache phase of migraines, to the exclusion of other neuropeptides. Moreover, according to Arulmozhi et al, (2005) the following has been identified in migraine sufferers: (1) a strong correlation between plasma CGRP concentrations and migraines; (2) the infusion of CGRP produced a migraine-like headache; (3) baseline CGRP levels were elevated; and (4) changes in plasma CGRP levels during migraine attacks significantly correlated with headache intensity. (Arulmozhi, D. K., et al., Vas. Pharma., 43: 176-187 (2005)).

In certain embodiments the invention relates to the CGRP binding antibody Eptinezumab with the following sequences

### Heavy Chain CDRs for Eptinezumab

SEQ ID No.: 1
CDR-H1: GYYMN
SEQ ID No.: 2
CDR-H2: VIGINGATYYASWAKG
SEQ ID No.: 3
CDR-H3: GDI

### The Variable Heavy Chain of Eptinezumab Comprises

SEQ ID No.: 4
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSS

The Heavy Chain of Eptinezumab comprises

SEQ ID No.: 5
EVQLVESGGG LVQPGGSLRL SCAVSGIDLS GYYMNWVRQA
PGKGLEWVGV IGINGATYYA SWAKGRFTIS RDNSKTTVYL
QMNSLRAEDT AVYFCARGDI WGQGTLVTVS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDARVE PKSCDKTHTC PPCPAPELLG GPSVFLFPPK
PKDTLMISRT PEVTCVVVDV SHEDPEVKFN WYVDGVEVHN
AKTKPREEQY ASTYRVVSVL TVLHQDWLNG KEYKCKVSNK
ALPAPIEKTI SKAKGQPREP QVYTLPPSRE EMTKNQVSLT
CLVKGFYPSD IAVEWESNGQ PENNYKTTPP VLDSDGSFFL
YSKLTVDKSR WQQGNVFSCS VMHEALHNHY TQKSLSLSPG

A C-terminal lysine (K) may be present in case the Heavy Chain is not processed in a system that cleave this, e.g. in a yeast such as Pichia, but the c-terminal lysine will usually be cleaved in a CHO expression system:

SEQ ID No.: 6
EVQLVESGGG LVQPGGSLRL SCAVSGIDLS GYYMNWVRQA
PGKGLEWVGV IGINGATYYA SWAKGRFTIS RDNSKTTVYL
QMNSLRAEDT AVYFCARGDI WGQGTLVTVS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDARVE PKSCDKTHTC PPCPAPELLG GPSVFLFPPK
PKDTLMISRT PEVTCVVVDV SHEDPEVKFN WYVDGVEVHN
AKTKPREEQY ASTYRVVSVL TVLHQDWLNG KEYKCKVSNK
ALPAPIEKTI SKAKGQPREP QVYTLPPSRE EMTKNQVSLT
CLVKGFYPSD IAVEWESNGQ PENNYKTTPP VLDSDGSFFL
YSKLTVDKSR WQQGNVFSCS VMHEALHNHY TQKSLSLSPGK

### Light Chain CDRs for Eptinezumab

SEQ ID No.: 7
CDR-L1: QASQSVYHNTYLA
SEQ ID No.: 8
CDR-L2: DASTLAS
SEQ ID No.: 9
CDR-L3: LGSYDCTNGDCFV

The Variable Light Chain of Eptinezumab comprises

SEQ ID No.: 10
QVLTQSPSSLSASVGDRVTINCQASQSVYHNTYLAWYQQKPGKVPKQLIY
DASTLASGVPSRFSGSGSGTDFTLTISSLQPEDVATYYCLGSYDCTNGDC
FVFGGGTKVEIKR

The Light Chain of Eptinezumab comprises

SEQ ID No.: 11
QVLTQSPSSL SASVGDRVTI NCQASQSVYH NTYLAWYQQK
PGKVPKQLIY DASTLASGVP SRFSGSGSGT DFTLTISSLQ
PEDVATYYCL GSYDCTNGDC FVFGGGTKVE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

According to one embodiment of the invention the Fc-containing monoclonal antibody is Eptinezumab or one comprising the same CDRs or variable regions as Eptinezumab.

The product according to the invention, e.g. Eptinezumab, may be expressed in a suitable cell line such as a CHO cell line using a bioprocess. It is important for the quality of the product that the harvest from the bioprocess (Harvest Cell Culture Fluid (HCCF)) is kept cold and at a certain pH to avoid e.g. deamidation and aggregation of the product before subjecting it to a down-stream purification process. The inventor of the current invention has found out that the optimal temperature is about 2-8° C., such as about 5° C., and a pH about 7.

Before contacting the resin in a chromatographic column with a load fluid, it may be necessary to adjust parameters such as pH, ionic strength, and temperature and in some instances the addition of substances of different kinds. Thus, it is an optional step to perform an equilibration of the chromatographic column by washing it with a solution (e.g., a buffer for adjusting pH, ionic strength, etc., or for the introduction of a detergent) bringing the necessary characteristics for binding and purification of the product.

According to one aspect of the invention the protein of interest (e.g. Eptinezumab) is first subjected to a Protein A purification step (run in a bind-elute mode), followed by a Cation Exchange step (run in a bind elute mode) and an Anion Exchange step (run in a flow-through mode) to remove the remaining impurities. In the below embodiments are examples of suitable conditions for running and binding Eptinezumab.

In one embodiment of the invention, the Protein A chromatographic column is equilibrated and washed with a wash solution, thereby bringing the necessary characteristics for purifying the product. In one embodiment of the invention, the Protein A chromatographic column may be equilibrated using a solution containing a salt, e.g., about 10 mM to about 30 mM (e.g. 20 mM) NaPO₄, and about 100 mM to about 150 mM (e.g. 100 mM) NaCl. The pH of the equilibration buffer may range from about 6.0 to about 7.0. In one embodiment, the pH of the equilibration buffer is about 6.8. After contacting the resin in the Protein A chromatographic column with the load fluid, such as conditioned culture medium described herein above and/or HCCF described herein above from a bioreactor or bioprocess with CHO cells expressing Eptinezumab, the resin is first washed. A first wash may according to one embodiment of the invention be with the above-mentioned equilibrium solution.

In accordance with the invention, following the equilibrium of the Protein A chromatographic column, and optionally a first wash without arginine or arginine salt or arginine derivative, a subsequent wash solution used in the method described herein may contain arginine or an arginine derivative. The arginine derivative can be, but is not limited to, acetyl arginine, agmatine, arginic acid, N-alpha-butyroyl-L-arginine, or N-alpha-pivaloyl arginine.

The concentration of arginine or arginine derivative in the wash solution is between about 500 mM and about 600 mM, e.g., 500 mM, 525 mM, 550 mM, 575 mM or 600 mM. In certain embodiments, the concentration of arginine or arginine derivative in the wash solution is between about 500 mM to about 600 mM or about 525 mM to about 575 mM, or about 550 mM to about 575 mM. In certain embodiments, the concentration of arginine or arginine derivative in the wash solution is greater than about 500 mM and less than about 600 mM.

The pH of the wash solution is generally between about 8.0 and about 8.7, for example, 8.1, 8.2, 8.3, 8.4, 8.5, 8.6, and 8.7. In some cases, the pH of the wash solution is greater than 8.0 and less than about 8.7. The wash solution may contain 20 mM to 50 mM sodium phosphate (Na₂HPO₄) (e.g., 20 mM, 30 mM, 40 mM or 50 mM). In one embodiment the bound medium is washed with 5 column volumes of the wash solution, followed by an elution step.

The wash step with arginine or arginine derivative, may subsequently be followed by one or more wash steps without arginine or arginine derivative. According to one embodiment this may be using a Sodium Acetate containing wash medium (e.g. at a sodium acetate concentration of about 20 mM to about 50 mM, such at 20 mM, 25 mM, 30 mM 40 mM or 50 mM) at a pH about 5 to about 6 (e.g. pH about 5.4).

In certain embodiments of the invention, the product may be eluted from the Protein A chromatographic column (in a bind-elute mode). To elute a product from the Protein A chromatographic column, the washed resin from said chromatographic column is contacted with an elution buffer. In some embodiments, the elution buffer contains about 15 mM to about 50 mM (e.g. 20 mM) Acetic Acid. In further embodiments, the elution buffer may also contain 20 mM to 50 mM (e.g. 30 mM) glycine. The pH of the elution buffer may range from about 2.0 to about 5.0. In one embodiment, the pH of the elution buffer is about 4.0. In another embodiment the pH of the buffer is about 3.65 or between 3.55 to 3.75.

The resin from the chromatographic column may optionally be cleaned, i.e., stripped and regenerated, after elution of the antibody. This procedure is typically performed regularly to minimize the building up of impurities on the surface of the solid phase and/or to sterilize the matrix to avoid contamination of the product with microorganisms.

Buffer components may be adjusted according to the knowledge of the person of ordinary skill in the art. Sample buffer composition ranges are provided in the Examples below. Not all of the buffers or steps are necessary but are provided for illustration only. A high throughput screen, as described in the Examples, may be used to efficiently optimize buffer conditions for Protein A column chromatography.

The eluate can include a product and the ratio of the product to host cell protein is increased compared to a corresponding method in which no detectable amount of arginine or arginine derivative is used in a wash solution.

In a subsequent step a Cation Exchange Chromatography may be applied. In the below embodiments are suitable conditions for running and binding Eptinezumab.

The Cation Exchange Chromatography may according to one embodiment of the invention be equilibrated, thereby bringing the necessary characteristics for purifying the product. In one embodiment of the invention, the Cation Exchange Chromatography column may be equilibrated using a solution containing a Sodium Acetate, such as about 10 mM to about 30 mM Sodium Acetate, e.g. 25 mM Sodium Acetate. The pH of the equilibration buffer may range from about 5.0 to about 6.0. In one embodiment, the pH of the equilibration buffer is about 5.4. After contacting the resin in a chromatographic column with the load fluid, the resin is first washed. A first wash may according to one embodiment of the invention be with bis-tris at a range of about 20-30 mM (e.g. 25 mM) and a pH about 7.

In accordance with the invention, following the equilibrium of the Cation Exchange Chromatography column, the product is eluted (in a bind-elute mode). To elute a product, the washed resin from said chromatographic column is contacted with an elution buffer. In some embodiments, the elution buffer contains Sodium Acetate and Tris buffer an about equal amounts, such as about 40% Sodium Acetate and about 60% Tris or 45% Sodium Acetate and 55% Tris buffer. Sodium Acetate concentration is about 20 mM to 30 mM (e.g. 25 mM) at a pH between 5 and 6 (e.g. pH 5.4), and the Tris buffer is between 90-110 mM Tris (e.g. 100 mM) at a pH about 7.

The resin from the chromatographic column may optionally be cleaned, i.e., stripped and regenerated, after elution of the antibody. This procedure is typically performed regularly to minimize the building up of impurities on the surface of the solid phase and/or to sterilize the matrix to avoid contamination of the product with microorganisms.

Buffer components may be adjusted according to the knowledge of the person of ordinary skill in the art. Sample buffer composition ranges are provided in the Examples below. Not all of the buffers or steps are necessary but are provided for illustration only. A high throughput screen, as described in the Examples, may be used to efficiently optimize buffer conditions for Cation Exchange Chromatography.

According to an aspect of the invention an Anion Exchange Chromatography step may be applied to an eluate from the Cation Exchange Chromatography. In the below embodiments are suitable conditions for running and binding Eptinezumab.

The Anion Exchange Chromatography may according to one embodiment of the invention be equilibrated, thereby bringing the necessary characteristics for purifying the product. In one embodiment of the invention, the Anion Exchange Chromatography column may be equilibrated using a solution containing a Tris, such as about 90 mM to about 110 mM Tris, e.g. 100 mM Tris. The pH of the equilibration buffer may be 7. After contacting the resin in a chromatographic column with the load fluid, the resin is washed. The Anion Exchange Chromatography is run in a flow-through mode. The washing may according to one embodiment of the invention be with Tris at a range of about 90 mM to about 110 mM Tris, e.g. 100 mM Tris. The pH of the equilibration buffer may be 7. Following the wash step, the chromatographic column may be stripped using a 10-30 mM (e.g. 20 mM) Tris and 0.2-0.8 mM (e.g. 0.5 mM) Sodium Chloride buffer.

The resin from the chromatographic column may optionally be cleaned, i.e., stripped and regenerated, after elution of the antibody. This procedure is typically performed regularly to minimize the building up of impurities on the surface of the solid phase and/or to sterilize the matrix to avoid contamination of the product with microorganisms.

Buffer components may be adjusted according to the knowledge of the person of ordinary skill in the art. Sample buffer composition ranges are provided in the Examples below. Not all of the buffers or steps are necessary but are provided for illustration only. A high throughput screen, as described in the Examples, may be used to efficiently optimize buffer conditions for Anion Exchange Chromatography

The present invention also relates to a product prepared according to a method described herein. In general, it will typically be desirable to further isolate and/or purify products isolated according to the present invention and formulate them for pharmaceutical use according to standard methods. For proteins, see for example Protein Purification Principles and Practice 2nd Edition, Springer-Verlag, New York, 1987; Higgins, S. J. and Hames, B. D. (eds.), and Deutscher, M. P., Simon, M. I., Abelson, J. N. (eds.), Guide to Protein Purification: Methods in Enzymology (Methods in Enzymology Series, Vol 182), Academic Press, 1997, incorporated herein by reference. One of ordinary skill in the art will appreciate that the exact techniques used will vary depending on the character of the product. Products of the invention having pharmacologic activity will be useful in the preparation of pharmaceuticals. These may be administered to a subject or may first be formulated for delivery by any available route including, but not limited to parenteral (e.g., intravenous), intradermal, subcutaneous, oral, nasal, bronchial, ophthalmic, transdermal (topical), transmucosal, rectal, and vaginal.

A pharmaceutical composition of the product is formulated to be compatible with its intended route of administration according to methods known in the art, see for example, Remington: The Science & Practice of Pharmacy”, 19th ed., Williams & Williams, (1995), and the “Physician's Desk Reference”, 52nd ed., Medical Economics, Montvale, N.J. (1998). In some embodiments, the product is formulated using sterile water (e.g., SWFI), buffered saline (e.g., phosphate buffered saline), polyol (e.g., glycerol, propylene glycol, liquid polyethylene glycol), or suitable mixtures thereof.

In a preferred embodiment the pharmaceutical composition comprises Eptinezumab in a highly pure form with 0.2-0.5% or 0.1-0.5% aggregates from proteins and/or peptides, 0.2-0.5 ppm or 0.1-0.5 ppm rPA and 0.2-0.5 ppm or 0.1-0.5 ppm HCP. In one embodiment rPA is determined using Cygnus Protein A ELISA Kits (F050H, F400, F400Z) or Protein A Mix-N-Go™ ELISA Kits (F600, F610, F740, F910, F950, F965). According to one embodiment aggregates are determined by size exclusion chromatography using HPLC. According to one embodiment host cell proteins are determined using ELISA HCP kits e.g. from Cygnus such as CHO HCP ELISA.

The pharmaceutical composition or process may further be characterized by the number of heavy chain and light chain MHC-associated peptides of Eptinezumab when subjected to an MHC-associated Peptide Proteomics (MAPPs) assay. The process of the invention and the pharmaceutical composition of the invention has less than a total of 240 different peptides from the heavy chain and less than a total of 60 different peptides from the light chain. These peptides may be identified or measured using MHC-associated Peptide Proteomics as described further in the Examples.

In accordance with the above, in a preferred embodiment, the pharmaceutical composition contains one or more (such as 1, 2 or 3) peptides of KTTVYLQMNSLRAEDTA (SEQ ID No.: 12), PEVKFNWYVD (SEQ ID NO.: 13) ALHNHYTQKSLSLSPG (SEQ ID NO.: 14) as HLA:peptide complexes from the heavy chain of Eptinezumab and/or one or more (such as 1, 2 or 3) of DRVTINCQASQSVYHNT (SEQ ID NO:15),VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18) as HLA:peptide complexes from the light chain of Eptinezumab when subjected to an MHC-associated Peptide Proteomics (MAPPs) assay. In another embodiment, the number of said peptides will be reduced as HLA:peptide complexes when subjected to an MHC-associated Peptide Proteomics (MAPPs) assay compared to a process using Pichia cells.

In a particular embodiment, the pharmaceutical composition does not contain the peptide DSKDSTYSLSSTLTLSKA (SEQ ID NO.: 19) subjected to an MHC-associated Peptide Proteomics (MAPPs) assay. In another embodiment said peptide is present in less than 5%, such as 1-5%, 1%, 2%, 3%, 4% or 5% of said peptide (SEQ ID NO.: 19). In another embodiment, the peptide DSKDSTYSLSSTLTLSKA (SEQ ID NO.: 19) will not be a HLA:peptide complex present in the pharmaceutical composition of the invention or will not be a HLA:peptide complex in the purified CHO cell process of the invention when subjected to an MHC-associated Peptide Proteomics (MAPPs) assay. In yet a further embodiment the HLA:peptide complex will be present in an amount of less than 5%, such as 1-5%, 1%, 2%, 3%, 4% or 5% compared to the total amount of HLA:peptide complexes measured in the MHC-associated Peptide Proteomics (MAPPs) assay. In still a further embodiment, the peptide DSKDSTYSLSSTLTLSKA (SEQ ID NO.: 19) will not be a HLA:peptide complex in the purified CHO cell process of the invention comprising the Protein A exchange chromatography, the Cation Exchange chromatography and the Anion Exchange Chromatography of the invention when subjected to an MHC-associated Peptide Proteomics (MAPPs) assay. In yet a further embodiment the HLA:peptide complex will be present in an amount of less than 5%, such as 1-5%, 1%, 2%, 3%, 4% or 5% compared to the total amount of HLA:peptide complexes in said CHO cell process.

Non-limiting examples of products that can be recovered using the methods described herein include a protein or a peptide, e.g., an antibody, an antibody fragment, a recombinant protein, a naturally secreted protein, a protein or a peptide that is engineered to be secreted, a non-protein product that is produced by a cell, or a combination of the foregoing products.

Further Embodiments of the invention include the following:

E1. A method for purifying Eptinezumab from a CHO bioprocess said method comprises an Anion Exchange Chromatographic step, wherein the Anion Exchange Chromatographic column is equilibrated in a buffer comprising about 90 mM to about 110 mM Tris, e.g. 100 mM Tris, pH 7.

E2. The method according to E1 wherein the Anion Exchange Chromatography is washed in a buffer comprising about 90 mM to about 110 mM Tris, e.g. 100 mM Tris, pH 7.

E3. The method according to E1 or E2, wherein the Anion Exchange Chromatography is stripped in a buffer comprising about 10 mM to about 30 mM (e.g. 20 mM) Tris and about 0.2-0.8 mM (e.g. 0.5 mM) Sodium Chloride, pH about 7 (e.g. pH 7.5).

E4. The method according to any of the previous Embodiments, wherein the Anion Exchange Chromatography is run in a flow-through mode.

E5. The method according to any of the previous Embodiments, wherein the Anion Exchange Chromatographic step results in an Eptinezumab product having 0.2-0.4% protein or peptide aggregates, and 0.6-0.2 ppm rPA and 0.15-0.16 ng/mg HCP.

E6. The method according to any of the previous Embodiments, wherein the Anion Exchange Chromatography is preceded by a Cation Exchange Chromatography step (run in a bind-elute mode).

E7. The method according to any of the previous Embodiments, wherein the Cation Exchange Chromatography is equilibrated in a solution comprising about 10 mM to about 30 mM Sodium Acetate, e.g. 25 mM Sodium Acetate, pH about 5.0 to about 6.0. such as about 5.4.

E8. The method according to any of previous Embodiments, wherein the Cation chromatographic column is washed in a bis-tris buffer at a range of about 20-30 mM (e.g. 25 mM), pH about 7 after contacting the resin with the load fluid.

E9. The method according to any of the previous Embodiments, wherein Cation Exchange Chromatography column is eluted using a elution buffer comprising Sodium Acetate and Tris buffer in an about equal amounts (such as about 40% Sodium Acetate and about 60% Tris or 45% Sodium Acetate and 55% Tris buffer) and the Sodium Acetate concentration is about 20 mM to 30 mM (e.g. 25 mM), pH between 5 and 6 (e.g. pH 5.4), and the Tris buffer is between 90-110 mM Tris (e.g. 100 mM), pH about 7.

E10. The method according to any of the previous Embodiments, wherein the first step in the method is a Protein A chromatographic column (run in a bind-elute mode).

E11. The method according to Embodiment E10, wherein the Protein A chromatographic column is equilibrated using a solution comprising a salt, e.g., about 10 mM to about 30 mM (e.g. 20 mM) NaPO₄, and about 100 mM to about 150 mM (e.g. 100 mM) NaCl, at a pH from about 6.0 to about 7.0 (e.g. pH 6.8).

E12. The method according to Embodiment E11, wherein the Protein A chromatographic column is washed using a wash solution comprising arginine concentration between about 500 mM to about 600 mM, e.g. 575 mM, pH about 8.5, and 20 mM to 50 mM sodium phosphate (Na₂HPO₄) (e.g., 20 mM, 25 mM, 30 mM, 40 mM or 50 mM).

E13. The method according to Embodiment E12, wherein a subsequent second wash step is applied using a Sodium Acetate containing wash medium at a Sodium Acetate concentration of about 20 mM to about 50 mM (such at 20 mM, 25 mM, 30 mM 40 mM or 50 mM) at a pH about 5 to about 6 (e.g. pH about 5.4).

E14. The method according to Embodiment E13, wherein the Protein A chromatographic column is contacted with an elution buffer that comprises about 15 mM to about 50 mM (e.g. 20 mM) Acetic Acid, 20 mM to 50 mM (e.g. 30 mM) glycine, pH about 3.65 or between 3.55 to 3.75.

E15. A pharmaceutical composition comprising Eptinezumab obtained by the method according to any one or all of Embodiments E1-E14.

E16. The method or the pharmaceutical composition according to any one of the previous Embodiments wherein Eptinezumab comprises all six CDR sequences
Light Chain CDR 1 SEQ ID NO.: 7, Light Chain CDR 2 SEQ ID NO.: 8, Light Chain CDR 3 SEQ ID NO.: 9, Heavy Chain CDR 1 SEQ ID NO.: 1, Heavy Chain CDR 2 SEQ ID NO.: 2, and Heavy Chain CDR 3 SEQ ID NO.: 3.

E17. The method or the pharmaceutical composition according to any one of the previous Embodiments wherein Eptinezumab has the VH region as defined in SEQ ID NO.: 4 and the and VL as defined in SEQ ID NO.: 10.

E18. The method or the pharmaceutical composition according to any one of the previous Embodiments wherein Eptinezumab has the heavy chain as defined in SEQ ID NO.: 5 or SEQ ID NO.: 6 and the light chain as defined in SEQ ID NO.: 11.

E19. A Pharmaceutical composition comprising Eptinezumab having a sequence in accordance with E14-E16, comprising 0.2-0.5% aggregates of Eptinezumab, 0.2-0.5 ppm rPA and 0.2-0.5 HCP.

E20. The method or the pharmaceutical composition according to any one of the previous Embodiments wherein the Cation Exchange chromatography step is effected using a resin, preferably a Capto S ImpAct resin, more preferably a resin composed of an agarose base comprising a polymer ligand of pyrrolidone and sulfonate.

E21: The method or the pharmaceutical composition according to any one of the previous Embodiments wherein the Anion Exchange Chromatography step is effected using a resin is composed of a base material of hydroxylated methacrylic polymer beads that have been functionalized with a primary amine, optionally a proprietary primary amine (NH2) strong anion exchange groups such as Toyopearl NH2-750F.

E22: The method or the pharmaceutical composition according to any one of the previous Embodiments wherein the Cation Exchange chromatography step is effected using a resin, preferably a Capto S ImpAct resin, e.g., a resin composed of an agarose base comprising a polymer ligand of pyrrolidone and sulfonate; and the Anion Exchange Chromatography is e.g., effected using a resin is composed of a base material of hydroxylated methacrylic polymer beads that have been functionalized with a primary amine, e.g., a proprietary primary amine (NH2) strong anion exchange groups such as Toyopearl NH2-750F.

E23. The method or the pharmaceutical composition according to any one of the previous Embodiments wherein the Eptinezumab composition comprises peptide fragments of Eptinezumab when subjected to an MHC-associated Peptide Proteomics (MAPPs) assay.

E24: The method or the pharmaceutical composition according to any one of the previous Embodiments wherein said peptides include one or more of KTTVYLQMNSLRAEDTA (SEQ ID No.: 12), PEVKFNWYVD (SEQ ID NO.: 13) ALHNHYTQKSLSLSPG (SEQ ID NO.: 14) from the heavy chain of Eptinezumab and/or one or more of DRVTINCQASQSVYHNT (SEQ ID NO:15), VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18) from the light chain of Eptinezumab when subjected to an MHC-associated Peptide Proteomics (MAPPs) assay.

E25: The method or the pharmaceutical composition according to any one of the previous Embodiments wherein said composition or pharmaceutical composition does not contain the peptide fragment of Eptinezumab: DSKDSTYSLSSTLTLSKA (SEQ ID NO.: 19), or e.g. contain less than 5%, such as 1-5%, 1%, 2%, 3%, 4% or 5%, when subjected to an MHC-associated Peptide Proteomics (MAPPs) assay.

E26: The method or the pharmaceutical composition according to any one of the previous Embodiments which includes the removal or reducing the amount or number of peptide impurities wherein said peptide impurities comprise fragments of Eptinezumab when subjected to an MHC-associated Peptide Proteomics (MAPPs) assay.

E27: The method or the pharmaceutical composition according to any one of the previous Embodiments which includes the removal or reducing the amount or number of peptide impurities wherein said peptides include one or more of KTTVYLQMNSLRAEDTA (SEQ ID No.: 12), PEVKFNWYVD (SEQ ID NO.: 13) ALHNHYTQKSLSLSPG (SEQ ID NO.: 14) from the heavy chain of Eptinezumab and/or one or more of DRVTINCQASQSVYHNT (SEQ ID NO: 15), VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18) from the light chain of Eptinezumab, when subjected to an MHC-associated Peptide Proteomics (MAPPs) assay

E28. The method or the pharmaceutical composition according to any one of the previous Embodiments, wherein said composition or pharmaceutical composition does not contain the peptide fragment of Eptinezumab: DSKDSTYSLSSTLTLSKA (SEQ ID NO.: 19) or e.g. contain less than 5%, such as 1-5%, 1%, 2%, 3%, 4% or 5%, when subjected to an MHC-associated Peptide Proteomics (MAPPs) assay

E29 The method or the pharmaceutical composition according to any one of the previous Embodiments, wherein said peptides fragments from Eptinezumab from the heavy chain of Eptinezumab is less than a total of 240 different peptides and said peptide fragments from the light chain is less than a total of 60 different peptides, when subjected to an MHC-associated Peptide Proteomics (MAPPs) assay

E30. The method or the pharmaceutical composition according to Embodiments E29, wherein said peptide fragments from the heavy chain of Eptinezumab and light chain of Eptinezumab are MHC-associated peptides.

E31 The method or the pharmaceutical composition according to Embodiments E29, wherein said peptide fragments from the heavy chain of Eptinezumab and light chain of Eptinezumab are identified by MHC-associated Peptide Proteomics.

E32. The method or the pharmaceutical composition according to any one of the previous Embodiments, wherein the host cell producing Eptinezumab is a Chinese Hamster Ovary (CHO) cell line.

E33. The method or the pharmaceutical composition according to any one of the previous Embodiments, wherein the purification comprises Protein A Chromatography followed by Cation Exchange Chromatography and Anion Exchange Chromatography.

E33. The method or the pharmaceutical composition according to any one of the previous Embodiments, wherein the pharmaceutical composition further comprises Histidine, Polysorbate 80 and Sorbitol.

E34: The method or the pharmaceutical composition according to any one of the previous Embodiments which includes the removal or reducing the amount or number of peptide impurities, wherein said peptide impurities are removed by one or more of filtration, chromatography, optionally one or more of reversed-phase HPLC, ion exchange chromatography, size exclusion chromatography, solid phase extraction, diethyl ether precipitation, immobilized metal affinity chromatography, gel permeation chromatography, centrifugal partition chromatography and/or immunoaffinity separation.

### EXAMPLES

### Example 1

To explore the effect of the Anion (AEX) and Cation Exchange Chromatography (CEX) when purifying Eptinezumab, the columns were loaded with material and HCP, rPA and aggregates of Eptinezumab were measured.

The load material on the Protein A column was Harvest Cell Culture Fluid harvested from an upstream bioprocess of CHO cells expressing Eptinezumab. The Protein A Chromatography was conducted as described herein below.

The CEX load material was the eluate from a Protein A column and the AEX load was the eluate from the CEX column. Both the CEX and AEX was run according to the process described herein below.

In the CEX load the initial values for rPA was in the range 1.0-2.3 ppm, and for Eptinezumab aggregates 1.3-1.4% aggregates for the different batches were tested (Table 1).

TABLE 1

| | Batch 1 | Batch 2 | Batch 3 |
|---|---|---|---|
| rPA | 2.3 | 1.0 | 1.5 |
| % Aggregates | 1.4 | 1.3 | 1.3 |

In the AEX load (the eluate from the CEX column) the initial for rPA it was in the range 0.6-0.9 ppm, and for Eptinezumab aggregates 0.9-1.1% aggregates for the different batches tested (Table 2).

TABLE 2

| | Batch 1 | Batch 2 | Batch 3 |
|---|---|---|---|
| rPA | 0.9 | 0.6 | 0.7 |
| % Aggregates | 0.9 | 1.0 | 1.1 |

The CEX column could reduce the rPA about 30% (1.0-2.3 ppm to 0.6-0.9 ppm) and a modest effect on Eptinezumab aggregates (Table 3 results after CEX run).

TABLE 3

| | Run | Run | Run | Run | Run | Run | Run | Run | Run | Run |
|---|---|---|---|---|---|---|---|---|---|---|
| | 1 | 2 | 3 | 4 | 5 | 6 | 7 | 8 | 9 | 10 |
| rPA | 0.8 | 0.9 | 0.6 | 0.7 | 1.2 | 0.8 | 0.5 | 0.8 | 0.5 | 0.4 |
| % Aggregates | 0.8 | 0.9 | 1.0 | 1.1 | 0.7 | 0.7 | 0.7 | 0.7 | 0.6 | 0.7 |

The AEX column reduced the values of these parameters to very low levels. After the AEX column the % Eptinezumab aggregates were reduced to 0.2-0.5%, and the rPA to about 0.5 ppm or below (Table 4 results after AEX run).

TABLE 4

| | Run | Run | Run | Run | Run | Run | Run | Run | Run | Run |
|---|---|---|---|---|---|---|---|---|---|---|
| | 1 | 2 | 3 | 4 | 5 | 6 | 7 | 8 | 9 | 10 |
| rPA | <0.5 | <0.5 | <0.5 | <0.5 | <0.5 | <0.5 | <0.5 | <0.5 | <0.5 | <0.5 |
| % Aggregates | 0.2 | 0.4 | 0.3 | 0.4 | 0.3 | 0.4 | 0.4 | 0.4 | 0.4 | 0.5 |

In a similar way the HCP values were assessed. Loading the CEX (Table 5) with a loading material from the Protein A column having about 8-12 ppm HCP, the CEX were able to reduce the HCP to below 1.9 ppm (Table 6), whereas the AEX could further reduce this to below 0.5 ppm.

TABLE 5
(CEX loading material)

| | Batch 1 | Batch 2 | Batch 3 |
|---|---|---|---|
| HCP ppm | 8 | 7 | 12 |

TABLE 6
(Results after CEX run)

| | Run | Run | Run | Run | Run | Run | Run | Run | Run | Run |
|---|---|---|---|---|---|---|---|---|---|---|
| | 1 | 2 | 3 | 4 | 5 | 6 | 7 | 8 | 9 | 10 |
| HCP ppm | <1.9 | <1.8 | <1.8 | <1.8 | <1.7 | <1.6 | <1.7 | <1.7 | <1.7 | <1.7 |

TABLE 7
(AEX loading material)

| | Batch 1 | Batch 2 | Batch 3 |
|---|---|---|---|
| HCP ppm | <1.8 | <1.8 | <1.8 |

TABLE 8
(Results after AEX run)

| | Run | Run | Run | Run | Run | Run | Run | Run | Run | Run |
|---|---|---|---|---|---|---|---|---|---|---|
| | 1 | 2 | 3 | 4 | 5 | 6 | 7 | 8 | 9 | 10 |
| HCP ppm | <0.5 | <0.5 | <0.5 | <0.5 | <0.5 | <0.5 | <0.5 | <0.5 | <0.5 | <0.5 |

### Methods

HCP and rPA was measured using commercially available ELISA assays such as ELISA HCP kits from Cygnus e.g. CHO HCP ELISA and Cygnus Protein A ELISA Kits (F050H, F400, F400Z) and Protein A Mix-N-Go™ ELISA Kits (F600, F610, F740, F910, F950, F965), whereas % Eptinezumab aggregates was determined using size exclusion chromatography using HPLC.

The Protein A, Cation Exchange Chromatography and Anion Exchange Chromatography were run under the following conditions.

### Protein A Chromatography Steps

The Protein A chromatography (MabSelect, SuRe LX, GE Healthcare) steps were run in a bind-elute mode and the conditions are set forth in Table 9.

TABLE 9

| | | Volume | Linear | |
|---|---|---|---|---|
| Sequence of | | Required | Velocity | Flow |
| Steps | Solution Description | (CV) | (cm/hr) | Direction |
| Water Rinse | (water for injection) | ≥2 | ≤250 | Down |
| Pre-Use | 0.2M Sodium Hydroxide | ≥3 | ≤240 | Down |
| Sanitization | | | | |
| Post- | 20 mM Sodium Phosphate, | ≥2 | ≤250 | Down |
| Sanitization | 100 mM Sodium Chloride, | | | |
| Flush | pH 6.8 | | | |
| Equilibration | 20 mM Sodium Phosphate, | ≥5 | ≤250 | Down |
| | 100 mM Sodium Chloride, | | | |
| | pH 6.8 | | | |
| Load | HCCF | As Needed | ≤150 | Down |
| Wash 1 | 20 mM Sodium Phosphate, | ≥5 | ≤250 | Down |
| | 100 mM Sodium Chloride, | | | |
| | pH 6.8 | | | |
| Wash 2 | 20 mM Sodium Phosphate, | ≥5 | ≤250 | Down |
| | 575 mM Arginine, pH 8.5 | | | |
| Wash 3 | 25 mM Sodium Acetate, pH | ≥5 | ≤250 | Down |
| | 5.4 | | | |
| Elution | 20 mM Acetic Acid, 30 mM | 5 | ≤250 | Down |
| | Glycine, pH 3.75 | | | |
| Strip | 100 mM Acetic Acid | ≥3 | ≤250 | Down |
| Rinse | 20 mM Sodium Phosphate, | ≥3 | ≤250 | Down |
| | 100 mM Sodium Chloride, | | | |
| | pH 6.8 | | | |
| Cleaning | 0.2M Sodium Hydroxide | ≥3 | ≤240 | Down |
| (CIP) | | | | |

### Cation Exchange Chromatography

The Protein A eluate is pH adjusted with 1M Glycine (pH 2) to pH 3.6 and held for over an hour before adjustment to pH 5.4 with 1M BisTris solution and subsequent depth filtration to prepare load for Cation Exchange chromatography.

The Cation Exchange (CaptoS Impact, GE Healthcare) steps were run in a bind-elute mode and the conditions are set forth in Table 10.

TABLE 10

| | | Volume | Linear | |
|---|---|---|---|---|
| Sequence of | | Required | Velocity | Flow |
| Steps | Solution Description | (CV) | (cm/hr) | Direction |
| Water Rinse | WFI | ≥3 | ≤150 | Down |
| Pre-Use | 0.5M Sodium Hydroxide | ≥3 | ≤108 | Down |
| Sanitization | | | | |
| Pre- | 100 mM Sodium Acetate, | ≥3 | ≤180 | Down |
| Equilibration | 1M Sodium Chloride, pH | | | |
| | 5.4 | | | |
| Equilibration 1 | 25 mM Sodium Acetate, | ≥5 | ≤180 | Down |
| | pH 5.4 | | | |
| Load | Depth Filtered Load | As | ≤180 | Down |
| | | Needed | | |
| Wash | 25 mM Bis-Tris, pH 7.0 | ≥3 | ≤180 | Down |
| Elution | A: 25 mM Sodium | ≥6 | ≤180 | Down |
| | Acetate, pH 5.4 | | | |
| | B: 100 mM Tris, pH 7.0 | | | |
| | (45% A, 55% B) | | | |
| Strip | 100 mM Tris, pH 7.0 | ≥3 | ≤180 | Down |
| Sanitization | 0.5M Sodium Hydroxide | ≥3 | ≤108 | Down |
| Rinse | 100 mM Sodium Acetate, | ≥3 | ≤180 | Down |
| | 1M Sodium Chloride, pH | | | |
| | 5.4 | | | |
| Storage | 2% Benzyl Alcohol, | ≥3 | ≤180 | Down |
| | 0.2M Sodium Acetate | | | |

### Anion Exchange Chromatography

Eluate from Cation exchange step is adjusted to pH 7 using BisTris prior to anion exchange chromatography. The Anion Exchange (Toyopeal NH2-750F, Tosoh) were run in a flow-through mode and the process steps are set forth in Table 11.

TABLE 11

| | | Volume | Linear | |
|---|---|---|---|---|
| Sequence of | | Required | Velocity | Flow |
| Steps | Solution Description | (CV) | (cm/hr) | Direction |
| Equilibration | 100 mM Tris, pH 7.0 | ≥5 | ≤300 | Down |
| Load | Conditioned AEX Load | As Needed | ≤300 | Down |
| Wash | 100 mM Tris, pH 7.0 | 5 | ≤300 | Down |
| Strip | 20 mM Tris, 0.5M Sodium | ≥3 | ≤300 | Down |
| | Chloride, pH 7.5 | | | |
| Sanitization | 0.5M Sodium Hydroxide | ≥3 | ≤300 | Down |
| Storage | 0.1M Sodium Hydroxide | ≥3 | ≤300 | Down |

### Example 2

### MHC-Associated Peptide Proteomics (MAPPs)

### MAPPS

MHC-associated Peptide Proteomics (MAPPs) is a specialized analytical technique designed to identify peptides that are presented by MHC class II molecules on antigen-presenting cells (APCs). This method can facilitate identifying impurities.

MAPPs assays can provide insights into how a therapeutic protein is taken up, processed, and presented by antigen-presenting cells. This information potentially may be used to determine whether post-translational modifications, sequence variants, or process-related impurities influence the way peptides are presented.

### MAPPs Workflow

MAPPS may be used in different formats or workflows. One of such MAPPS workflows is described in Rombach-Riefgraf et al., 2014 PLoS ONE 9(1): e86322 (incorporated by reference herein). The assay can be divided into four main stages:

### PBMC Isolation

Peripheral blood mononuclear cells (PBMCs) are collected from healthy donors. Monocytes are then isolated using immunomagnetic separation, yielding a highly enriched population. These monocytes are cultured in a differentiation medium to generate monocyte-derived dendritic cells (moDCs).

### Therapeutic Protein Exposure

The semi-mature moDCs are exposed to the full-length therapeutic protein. The cells naturally process the protein, breaking it down into peptide fragments and presenting them on MHC class II molecules.

### Purification of MHC-Peptide Complexes

Mature moDCs are harvested, and the MHC-peptide complexes are isolated through immunoprecipitation techniques.

### Peptide Elution and Mass Spectrometry Analysis

Peptides bound to MHC molecules are eluted under acidic conditions and analyzed using high-resolution mass spectrometry. This step reveals the exact sequences of the presented peptides, thereby providing direct information as to potential T cell epitopes that may elicit an immune response in vivo.

The assay used in this Example is similar to the described in Rombach-Riefgraf et al., 2014 PLoS ONE 9(1): e86322 and is described below.

### MAPPS Assay Used to Identify Impurities of Eptinezumab

Six different production batches of Eptinezumab were tested using samples from 20 individual donors. Three of these batches were produced using a Pichia pastoris expression system (Process A), while the remaining three were produced using a CHO cell-based process (Process B), which represents the method of the present invention.

Monocyte derived dendritic cells (moDCs) from each donor were individually treated with each batch at a concentration of 100 μg/mL of eptinezumab, in the presence of lipopolysaccharide (LPS), for 24 hours. Following incubation, the moDCs were harvested, lysed, and their membrane fractions were isolated.

HLA-DR:peptide complexes were purified via immunopurification. The bound peptides were then eluted, sequenced using mass spectrometry, and mapped to the amino acid sequences of the eptinezumab heavy chain (HC) and light chain (LC). Human serum albumin, present in the assay medium, served as an internal control in all samples.

### Results

TABLE 12
Total Number of Peptides Identified by Process

| Protein sequence | Process A (Pichia) | Process B (CHO) |
|---|---|---|
| Albumin | 395 | 376 |
| Eptinezumab Heavy Chain | 410 | 243 |
| Eptinezumab Light Chain | 193 | 60 |

Table 12 summarizes the total number of peptides identified from each process, specifically for the heavy and light chains of eptinezumab. Peptides derived from human albumin were consistently detected across both processes, demonstrating good intra-donor reproducibility.

Notably, batches produced via Process A (Pichia) yielded a significantly average higher number of peptides, i.e., a 1.7-fold increase for the heavy chain and a 3.2-fold increase for the light chain compared to Process B (CHO).

A more detailed interrogation of the data indicates that in the Eptinezumab HC the 3 core immunodominant epitopes are: KTTVYLQMNSLRAEDTA (SEQ ID NO:12), PEVKFNWYVD (SEQ ID NO.: 13) and ALHNHYTQKSLSLSPG (SEQ ID NO.: 14). These 3 peptides are identified in both Process A and Process B batches of Eptinezumab but the Process A batches yield significantly more peptides across these 3 regions than Process B. The Light chain of Eptinezumab had 4 core immunodominant epitopes where the Process A batches yielded significantly more of these peptides: DRVTINCQASQSVYHNT (SEQ ID NO: 15), VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18). These 4 peptides were identified in both Process A and Process B batches of Eptinezumab, but the Process A batches yield significantly more peptides across these 4 regions than Process B. In addition, there are an additional peptide identified in Process A that are not found in Process B samples: DSKDSTYSLSSTLTLSKA (SEQ ID NO.: 19).

These findings suggest that eptinezumab produced using the Pichia process undergoes different post-translational modifications, resulting in a greater number of presented peptides. This highlights how the production method can influence antigen processing and presentation.

### Methods MAPPs Assay

### Isolation of Monocytes.

Donors were recruited at Phase I clinical trial units in the UK. All samples were collected under an ethical protocol approved by a local REC (research ethics committee) and written informed consent was obtained from each donor prior to sample donation. PBMC from healthy donors were prepared from whole blood within six hours of blood withdrawal. Cells are cryopreserved in vapor phase nitrogen until use in the assays. The quality and functionality of each PBMC preparation was analyzed using 7 day activation with positive controls such as KLH to assess naïve T cell responses.

The isolated PBMCs were labeled with human anti-CD14 microbeads (Miltenyi Biotec). After filtration of labeled cells through pre-separation filters, cells were magnetically separated with in a magnetic field.

Differentiation of Monocytes to Immature moDCs.

Monocytes were seeded into T12.5 flasks with 5-10e6 monocytes per flask in differentiation medium (medium containing GM-CSF and IL-4) and incubated for 5 days at 37° C., 5% CO2 to differentiate into immature moDC.

Stimulation and Loading of Immature moDCs.

After 5 days of cell culture, the moDC were then loaded with the process sample (A or B) containing Eptinezumab (100 mg/ml) or Albumin (control) and matured with LPS for 24 hours at 37° C. and 5% CO2, moDCs were harvested and washed in PBS.

### Isolation of HLA-DR Associated and Analysis of Peptides.

After maturation the moDC were lysed and the membrane fraction containing the HLA:peptide complexes was solubilized and incubated with Protein A mag Sepharose beads (GE Healthcare) coated with anti-HLA-DR antibody (Lonza) at 4° C. overnight. The following morning the beads were washed in TBS and the peptides eluted from the HLA-DR complex with 0.1% trifluoracetic acid. Finally, the peptides were purified by passing through a 10 kDa MWCO spin column and stored at −80° C. for LC/MS analysis.

What is claimed is:

1. A method for reducing impurities in an eluate comprising a product, the method comprising:
(a) providing a load fluid comprising a product and one or more impurities, wherein the product is an Fc-containing monoclonal antibody,
(b) applying the load fluid in a) to a Protein A chromatography column under conditions suitable for binding the product,
(c) applying the load fluid from b) to a Cation Exchange chromatography column under conditions suitable for binding the product,
(d) applying the load fluid from c) to an Anion Exchange chromatography column under conditions suitable for binding impurities,
to reduce the percentage of aggregates to 0.2-0.5% or 0.1-0.5%.

2. The method according to claim 1, wherein (i) residual protein A (rPA) is reduced to 0.2-0.5 ppm or 0.1-0.5 ppm and/or (ii) wherein host cell protein (HCP) is reduced to 0.2-0.5 ppm or 0.1-0.5 ppm.

3. (canceled)

4. The method according to claim 1, wherein
(i) the Protein A chromatographic column is equilibrated using a solution comprising a salt, optionally about 10 mM to about 30 mM (e.g. 20 mM) NaPO₄, and about 100 mM to about 150 mM (e.g. 100 mM) NaCl, at a pH from about 6.0 to about 7.0 (optionally pH 6.8);
(ii) the Protein A chromatographic column is washed using a wash solution comprising arginine concentration between about 500 mM to about 600 mM, optionally 575 mM, pH about 8.5, and 20 mM to 50 mM sodium phosphate (Na₂HPO₄) (optionally 20 mM, 25 mM, 30 mM, 40 mM or 50 mM);
(iii) a subsequent second wash step is applied to the Protein A chromatographic column using a Sodium Acetate containing wash medium at a Sodium Acetate concentration of about 20 mM to about 50 mM (optionally at 20 mM, 25 mM, 30 mM 40 mM or 50 mM) at a pH about 5 to about 6 (e.g. pH about 5.4);
(iv) the Protein A chromatographic column is contacted with an elution buffer that comprises about 15 mM to about 50 mM (optionally 20 mM) Acetic Acid, 20 mM to 50 mM (optionally 30 mM) glycine, pH about 3.65 or between 3.55 to 3.75;
(v) the Cation Exchange Chromatography is equilibrated in a solution comprising about 10 mM to about 30 mM Sodium Acetate, e.g. 25 mM Sodium Acetate, pH about 5.0 to about 6.0, optionally about 5.4;
(vi) the Cation chromatographic column is washed in a Bis-Tris buffer at a range of about 20-30 mM (optionally 25 mM), pH about 7 after contacting the resin with the load fluid;
(vii) the Cation Exchange Chromatography column is eluted using a elution buffer comprising Sodium Acetate and Tris buffer in about equal amounts (optionally about 40% Sodium Acetate and about 60% Tris or 45% Sodium Acetate and 55% Tris buffer) and the Sodium Acetate concentration is about 20 mM to 30 mM (optionally 25 mM), pH between 5 and 6 (optionally pH 5.4), and the Tris buffer is between 90-110 mM Tris (optionally 100 mM), pH about 7;
(viii) the Anion Exchange Chromatography is washed in a buffer comprising about 90 mM to about 110 mM Tris, optionally 100 mM Tris, pH 7;
(ix) the Anion Exchange Chromatography is stripped in a buffer comprising about 10 mM to about 30 mM (optionally 20 mM) Tris and about 0.2-0.8 mM (optionally 0.5 mM) Sodium Chloride, pH about 7 (optionally pH 7.5);
(x) the Anion Exchange Chromatography is run in a flow-through mode; or
(xi) any combination of one or more of (i) to (x).

5-13. (canceled)

14. The method according to claim 1, which further includes the removal or reducing the amount or number of peptide impurities wherein said peptide impurities comprise fragments of Eptinezumab as measured in an MHC-associated Peptide Proteomics (MAPPs) assay.

15. The method of claim 14, wherein said peptide impurities peptides include one or more of KTTVYLQMNSLRAEDTA (SEQ ID No.: 12), PEVKFNWYVD (SEQ ID NO.: 13) ALHNHYTQKSLSLSPG (SEQ ID NO.: 14) from the heavy chain of Eptinezumab and/or one or more of DRVTINCQASQSVYHNT (SEQ ID NO:15), VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18) from the light chain of Eptinezumab as measured in an MHC-associated Peptide Proteomics (MAPPs) assay.

16. The method of claim 14, wherein said peptide impurities are removed by one or more of filtration, chromatography, optionally one or more of reversed-phase HPLC, ion exchange chromatography, size exclusion chromatography, solid phase extraction, diethyl ether precipitation, immobilized metal affinity chromatography, gel permeation chromatography, centrifugal partition chromatography and/or immunoaffinity separation.

17. A pharmaceutical composition comprising Eptinezumab obtained by the method according to claim 1.

18. The method er the pharmaceutical composition according to claim 1 wherein the Fc-containing monoclonal antibody is Eptinezumab or an Fc-containing monoclonal antibody comprising the CDR sequences of Eptinezumab which comprise:
Light Chain CDR 1 SEQ ID NO.: 7, Light Chain CDR 2 SEQ ID NO.: 8, Light Chain CDR 3 SEQ ID NO.: 9, and
Heavy Chain CDR 1 SEQ ID NO.: 1, Heavy Chain CDR 2 SEQ ID NO.: 2, and Heavy Chain CDR 3 SEQ ID NO.: 3.

19. The method according to claim 1 wherein (i) Eptinezumab has the VH region as defined in SEQ ID NO.: 4 and the and VL as defined in SEQ ID NO.: 10; and/or
(ii) Eptinezumab has the heavy chain as defined in SEQ ID NO.: 5 or SEQ ID NO.: 6 and the light chain as defined in SEQ ID NO.: 11.

20. (canceled)

21. A pharmaceutical composition comprising Eptinezumab according to claim 17, comprising 0.2-0.5% or 0.1-0.5% aggregates of Eptinezumab, 0.2-0.5 ppm or 0.1-0.5 ppm rPA and 0.2-0.5 ppm or 0.1-0.5 ppm HCP.

22. The method according to claim 1 wherein
(i) the Cation Exchange chromatography is effected using a resin, optionally, a Capto S ImpAct resin, optionally, a resin composed of an agarose base comprising a polymer ligand of pyrrolidone and sulfonate-;
(ii) the Anion Exchange Chromatography is effected using a resin composed of a base material of hydroxylated methacrylic polymer beads that have been functionalized with a primary amine, optionally one comprising proprietary primary amine (NH2) strong anion exchange groups such as Toyopearl NH2-750F;
(iii) the Cation Exchange chromatography is effected using a resin, optionally a Capto S ImpAct resin, optionally a resin composed of an agarose base comprising a polymer ligand of pyrrolidone and sulfonate; and the Anion Exchange Chromatography is effected using a resin is composed of a base material of hydroxylated methacrylic polymer beads that have been functionalized with a primary amine, e.g., one comprising proprietary primary amine (NH2) strong anion exchange groups such as Toyopearl NH2-750F; or
a combination of one or more of (i) to (iii).

23-24. (canceled)

25. A composition produced according to claim 1 which comprises peptide fragments of the light chain and heavy chain of Eptinezumab as measured in an MHC-associated Peptide Proteomics (MAPPs) assay.

26. The Eptinezumab composition according to claim 17, which optionally comprises peptide fragments of the light chain and heavy chain of Eptinezumab wherein said peptides include one or more of KTTVYLQMNSLRAEDTA (SEQ ID No.: 12, PEVKFNWYVD (SEQ ID NO.: 13) ALHNHYTQKSLSLSPG (SEQ ID NO.: 14) from the heavy chain of Eptinezumab and/or one or more of DRVTINCQASQSVYHNT (SEQ ID NO: 15), VPKQLIYDASTLASGVPS (SEQ ID NO.: 16), DNALQSGNSQESVTEQDSK (SEQ ID NO.: 17) and KHKVYACEVTHQGLSSP (SEQ ID NO.: 18) as measured in an MHC-associated Peptide Proteomics (MAPPs) assay.

27. The Eptinezumab composition according to claim 17, wherein said composition does not contain the peptide DSKDSTYSLSSTLTLSKA (SEQ ID NO.: 19) as measured in an MHC-associated Peptide Proteomics (MAPPs) assay.

28. The Eptinezumab composition according to claim 17, wherein the number of said peptide fragments from the heavy chain of Eptinezumab is less or equal to a total of 240 different peptides and/or the number of said peptide fragments from the light chain is less or equal to a total of 60 different peptides as measured in an MHC-associated Peptide Proteomics (MAPPs) assay.

29. The Eptinezumab composition of claim 28, wherein said peptides from the heavy chain of Eptinezumab and/or light chain of Eptinezumab are MHC-associated peptides.

30. The Eptinezumab composition of claim 28, wherein said peptides from the heavy chain of Eptinezumab and/or light chain of Eptinezumab are identified by MHC-associated Peptide Proteomics.

31. The method according to claim 1 wherein Eptinezumab is from CHO cells expressing Eptinezumab.

32. The method according to claim 1, wherein the load fluid in claim 1 step a) is conditioned culture medium and/or harvested cell culture fluid (HCCF) from a bioreactor or bioprocess comprising Eptinezumab.
